(19) United States
(12) Patent Application Publication
(10) Pub. No.: US 2022/0378297 A1
(43) Pub. Date: Dec. 1, 2022

(54) SYSTEM FOR MONITORING NEURODEGENERATIVE DISORDERS THROUGH ASSESSMENTS IN DAILY LIFE SETTINGS THAT COMBINE BOTH NON-MOTOR AND MOTOR FACTORS IN ITS DETERMINATION OF THE DISEASE STATE

(71) Applicant: Echowear LLC, West Warwick, RI (US)

(72) Inventors: Nicholas Constant, West Warwick, RI (US); Kunal Mankodiya, Sharon, MA (US)

(73) Assignee: Echowear LLC, West Warwick, RI (US)

(21) Appl. No.: 17/752,309

(22) Filed: May 24, 2022

Related U.S. Application Data
(60) Provisional application No. 63/193,978, filed on May 27, 2021.

(51) Int. Cl. A61B 5/0205; A61B 5/024; G16H 40/63; A61B 5/11; A61B 5/00

(52) U.S. Cl. CPC A61B 5/0205; A61B 5/02405; G16H 40/63; A61B 5/1118; A61B 5/4815; A61B 5/02438; A61B 5/4812; A61B 5/4809; A61B 5/6802

(57) ABSTRACT

The method of the present invention quantifies the severity of a subject's neurodegenerative disorder. The subject answers a questionnaire which results in a patient-reported outcome dataset. Benchmark tests are carried out by the subject performing one or more tasks resulting in a task result dataset. Continuous sensors collect data resulting in a sensor dataset. Short assessment tests of the subject are conducted resulting in a short assessment dataset. The patient-reported outcome dataset, task result dataset, sensor dataset, and short assessment dataset are aggregated into an output dataset that includes non-motor outcome measures and motor outcome measures. A single score is generated that quantifies the severity of a neurodegenerative disorder of the subject based on the output dataset.

### CROSS REFERENCE TO RELATED APPLICATION

This application is related to, and claims benefit from, U.S. Provisional Application No. 63/193,978, filed on May 27, 2021, entitled “A METHOD TO ENABLE THE INTERNET OF THINGS TO ASSIST IN THE REPORTING OF SYMPTOMS, DISEASE SPECIFIC EXPERIENCES, AND DAILY LIFE ACTIVITIES,” incorporated by reference in its entirety, herein.

### BACKGROUND OF THE INVENTION

The present invention generally relates to systems for monitoring neurodegenerative disorders and the use of wearable devices for monitoring such disorders to better understand the state of the disease or disorder to assist in treatment thereof.

Patients with a chronic disease or disorder typically cannot easily identify appropriate information about their condition and state of being. Such individuals monitoring their disease, such as a caregiver or the patient themselves, struggle to trust the standalone reported experience of the patient with the disease.

Wearable devices, such as smart watches, activity trackers, and the like, have proliferated the market primarily in the health and wellness area but also in the medical domain such as pharmaceutical studies and treatment outcomes measurements. Furthermore, the internet and other accessible data stores have accumulated vast amounts of information of medical interest.

Patients with these neurodegenerative diseases commonly interact with these various electronic devices but struggle to make use of the vast amounts of generated data such as data relating to spatial, behavioral, biological, and other characteristics. Monitoring the long-term progression of the disease can outlast various technologies and patient-reported outcomes of treatments, interventions, and therapies often depend upon self-reporting directly from the patient. However, such reported outcomes are often unreliable, inaccurate, and subjective. Moreover, the results of any digital assessment require the results to be explainable and understandable.

However, there is no known solution to obtain this valuable patient data in an individually specific fashion. The collected data has not been efficiently collected, presented and explained in a trusted way to effectively help monitor the health and wellness of the patient wearing the wearable data collection device.

Also, the prior art is devoid of a solution to monitor neurodegenerative disorders and diseases through assessments in daily life settings that combine both non-motor and motor factors in its determination of the disease state, which would provide a more complete insight into the state of the patient's disorder.

There have been many attempts in the prior art to make various assessments to better understand the state of a patient's disorder or disease; however, none of them have been adequate to provide an accurate and meaningful assessment of a patient's daily function.

In view of the foregoing, there is a need for a more accurate and complete system and method to monitor neurodegenerative disorders and diseases.

### SUMMARY OF THE INVENTION

The present invention preserves the advantages of prior art systems and methods for monitoring neurodegenerative diseases and disorders and determining the state of the disease. In addition, it provides new advantages not found in currently available systems and methods and overcomes many disadvantages of such currently available systems, devices and methods.

The invention is generally directed to the novel and unique system and methods for monitoring neurodegenerative diseases and disorders and determining the state of the disease.

The system and method of the present invention aggregates data across various ubiquitous daily use technologies including wearables, such as smartwatches, smart rings, smart clothing, smart shoes, and the like, as well as ambient sensing devices, such as smart mats (bathroom or doormat), smart mattress and bedding, smart chairs, sofas, and the like. Other technologies currently available and suitable for use with the present invention also include ecological and environmental sensors such as thermostats, security cameras, weather sensors, and social.

The present invention identifies current trusted information stores for presentation to a person monitoring the disease for further evaluation and action, such as treatment. The present invention integrates the sensed data with the patient reported outcomes, which is further integrated into a secure network for sharing the information. For example, the data may be integrated into a secure network for viewing information for patients, doctors, medical team(s), hospital organizations, and any others that need to view the data.

The method of the present invention quantifies the severity of a subject's neurodegenerative disorder with a single score representing the state of the disorder. The subject answers a questionnaire which results in a patient-reported outcome dataset. Benchmark tests are carried out by the subject performing one or more tasks resulting in a task result dataset. Continuous monitoring is carried out by sensors to collect data resulting in a sensor dataset. Short assessment tests of the subject are conducted resulting in a short assessment dataset. The patient-reported outcome dataset, task result dataset, sensor dataset, and short assessment dataset are aggregated into an output dataset that uniquely includes non-motor outcome measures and motor outcome measures. A single score is generated that quantifies the severity of a neurodegenerative disorder of the subject based on the output dataset.

It is therefore an object of the present invention to provide a system and methods for monitoring neurodegenerative diseases and disorders.

It is a further an object of the present invention to provide a system and methods for monitoring neurodegenerative diseases and disorders that aggregates a patient-reported outcome dataset, task result dataset, sensor dataset, and short assessment dataset into an output dataset that includes non-motor outcome measures and motor outcome measures. The reported outcomes may be of any type including patient reported, observer reported, clinical reported, and device reported. The patient-reported, or self-reported, measures are captured in the form of a questionnaire. The questionnaires are scored by applying a predetermined point system to the patient's responses. Although self-report measures seem subjective in nature, self-report measures objectify a patient's perception. Device-reported, or performance-based, measures require the patient to perform a set of movements or tasks. These scores for performance-based measures can be based on either an objective measurement (e.g., time to complete a task) or a qualitative assessment that is assigned a score (e.g., normal or abnormal mechanics for a given task). Performance-based measures tend to bring to light physiologic factors. Patient reported outcome measures may capture a patient's perception, beliefs, social factors and/or health factors. Observer-reported measures are measurements completed by a parent, caregiver or someone who regularly observes the patient on a daily basis. Clinician-reported measures are measurements that are completed by a healthcare professional. The professional uses clinical judgement and reports on patient behaviors or signs that are observed by the professional.

It is yet another object of the present invention to provide a single score that is generated that quantifies the severity of a neurodegenerative disorder of the subject based on the output dataset.

### BRIEF DESCRIPTION OF THE DRAWING FIGURES

The novel features which are characteristic of the present invention are set forth in the appended claims. However, the invention's preferred embodiments, together with further objects and attendant advantages, will be best understood by reference to the following detailed description taken in connection with the accompanying drawings in which:

FIG. 1 is an overview of the wellness concerns of a patient with a neurodegenerative disorder or disease and how the system and method of the present invention interacts therewith;

FIG. 2 shows various non-motor related components that can be queried in accordance with the present invention;

FIG. 3 shows various motor related components that can be queried in accordance with the present invention;

FIG. 4 shows the elements of perception, capacity, and performance that can be queried as part of developing a single score representing the state of the disorder;

FIGS. 5A-C show examples of the questionnaires in accordance with the present invention;

FIGS. 6A-G show examples of tasks for benchmark assessments;

FIG. 7 shows various example devices, such as wearables and smartphones, for continuous monitoring of the patient; and

FIG. 8 shows a short assessment query.

### DETAILED DESCRIPTION OF THE PREFERRED EMBODIMENT

Certain exemplary embodiments will now be described to provide an overall understanding of the principles of the structure, function, manufacture, and use of the device and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present disclosure. Further, in the present disclosure, like-numbered components of the embodiments generally have similar features, and thus within a particular embodiment each feature of each like-numbered component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Further, to the extent that directional terms like proximal, distal, top, bottom, up, or down are used, they are not intended to limit the systems, devices, and methods disclosed herein. A person skilled in the art will recognize that these terms are merely relative to the system and device being discussed and are not universal. The present invention is related to monitoring neurodegenerative disorders and diseases, such as Parkinson's disease, Alzheimer's disease, Huntington's disease, amyotrophic lateral sclerosis (ALS), motor neuron disease, ataxia, multiple system atrophy (MSA), and other disorders and diseases. Further, for ease of discussion and by way of example, the present invention is discussed in detail herein in connection with Parkinson's disease but it should be understood that the present invention is not limited to Parkinson's disease and has applicability in any other disease or disorder.

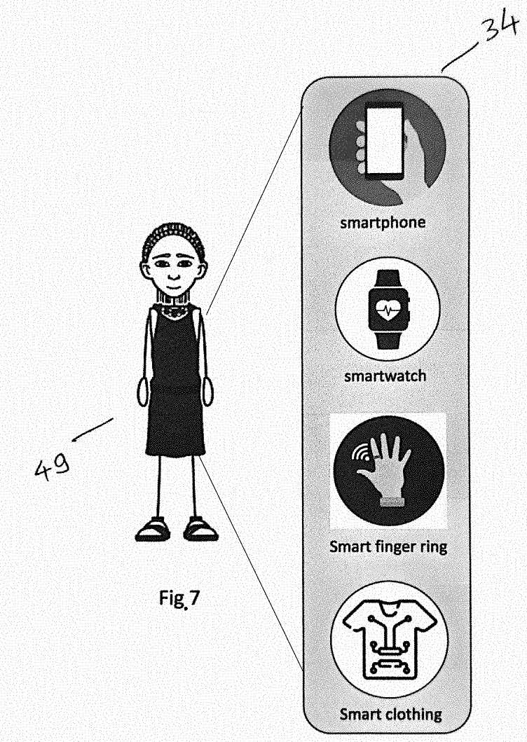

In general, the invention enables capacity, performance, and perception elements of an individual's health to be measured through a combination of subjective measures derived from the individual, capacity measures derived from the wearables 34, as in FIG. 7, symptom monitoring and their complement being performance for routinely managing the day's activities.

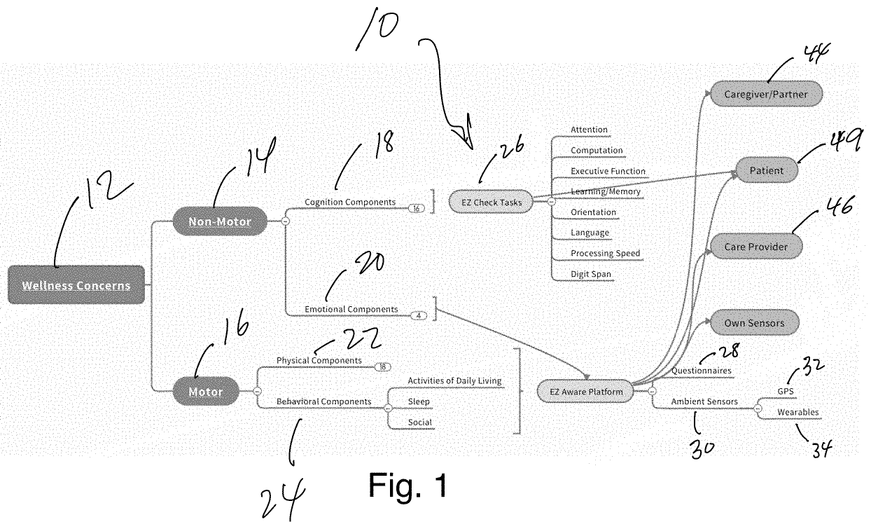

The system and method of the present invention provided a Patient-Centered Digital Health Platform 10 for Patient-Reported Outcomes (PROs) and Symptom Diary to address various wellness concerns 12, as shown in FIG. 1. The present invention allows a patient's voice to be heard through open response sections capable of categorical, textual and audio recordings. The patient is able to match symptoms to thoughts and feelings about the experience near the moments of symptomatic variations.

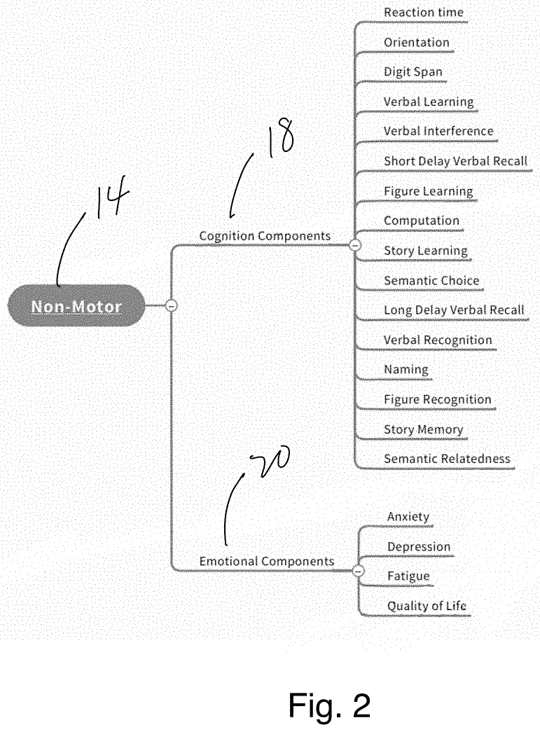

The wellness concerns 12 involve non-motor 14 and motor 16 elements. As seen in FIG. 2, the non-motor element 14 includes cognition components 18 of reaction time, orientation, digit span, verbal learning, short delay verbal recall, figure learning, computation, story learning, semantic choice, long delay verbal recall, verbal recognition, naming, figure recognition, story memory, and semantic relatedness, and the like. The non-motor element 14 also includes the emotional components 20 of anxiety, depression, fatigue, quality of life, and the like.

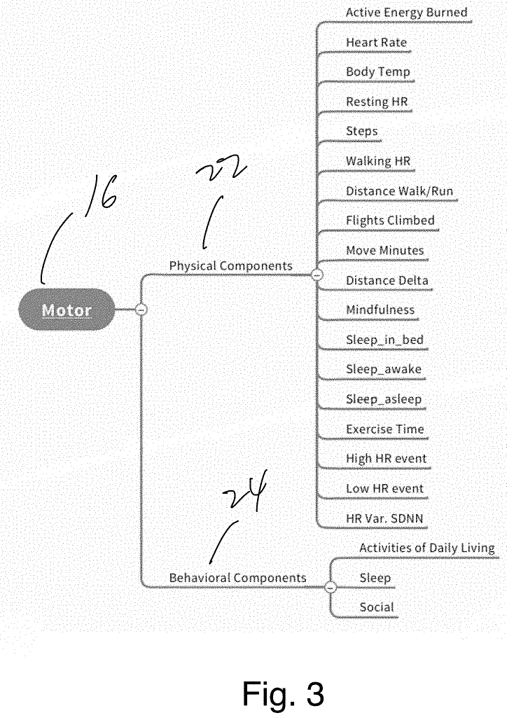

As seen in FIG. 3, the motor element 16 includes the physical components 22 of active energy burned, heart rate, body temperature, resting heart rate, steps, walking heart rate, distance of walk/run, flights climbed, move minutes, distance delta, mindfulness, sleep (in bed), sleep (awake), sleep (asleep), exercise time, high heart rate event, low heart rate event, heart rate variable/standard deviation of NN intervals (SDNN), and the like. The motor element 16 also includes the behavioral components 24 of activities of daily living, sleep, social, and the like.

The tasks 26 performed in accordance with the present invention can be to assess attention, computation, executive function, learning/memory, orientation, language, processing speed, digit span, and the like. Thus, the platform 10 can include questionnaires 28, ambient sensors 30, which may be of the nature of GPS 32 and wearables 34, for example, as shown in FIG. 1. Also, the platform 10 may be utilized and interacted with by the caregiver/partner of the patient, the patient themselves, the care provider, and with the patient's own sensors.

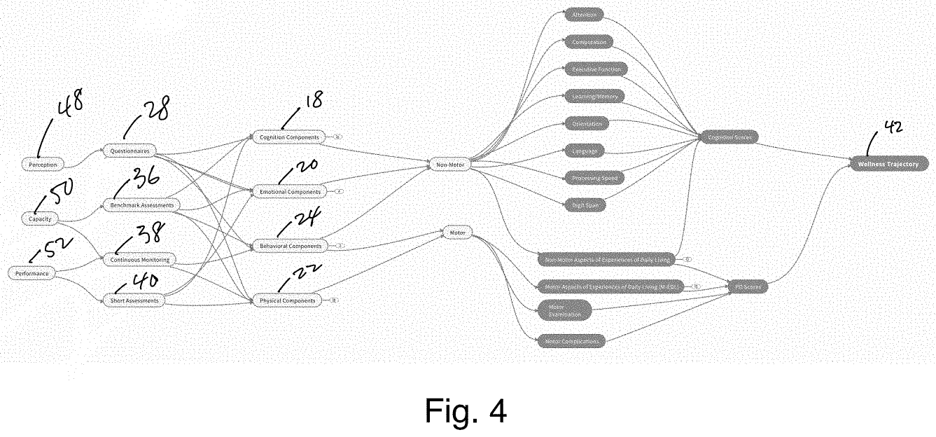

Turning now to FIG. 4, an overview of the system and method of the present invention 10 is shown to illustrate the aggregation of questionnaires 28, benchmark assessments 36, continuous monitoring 38 and short assessments 40 to arrive at a single disease state score 42.

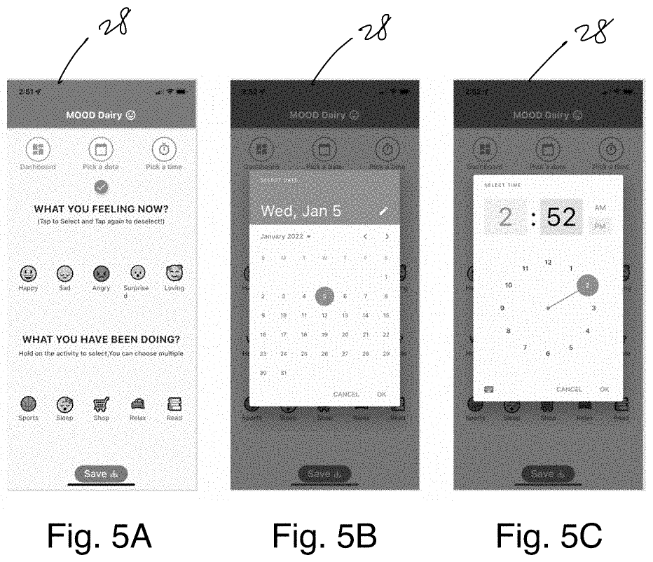

Questionnaires 28 via Patient-Reported Outcomes (PRO) are seen in FIGS. 5A-C. These PROs may include but are not limited to PD Questionnaire 39 (PDQ39), Beck Depression Inventory (BDI), Generalized Anxiety Disorder-7 (GAD7), Apathy Scale (AS), Fatigue Severity Scale (FSS), Activity of Daily Living Questionnaire (ADLQ), Parkinson's Disease Sleep Scale (PDSS), Caregiver Assessment, Unified Parkinson's Disease Rating Scale (UPDRS) Part I and 2. The PROs are managed through approved questionnaires 28 that need to be filled by patients in daily life settings. The system and method 10 of the present invention not only digitizes the process of PRO delivery through patient-centered interfaces, but also contextualizes PROs by leveraging the fusion of wearable sensors (e.g., smartwatches), IoT, and artificial intelligence, as will be discussed in detail below. For example, if the patient with Parkinson's Disease experiences a poor sleep quality, the system and method 10 of the present invention will learn the context of poor sleep quality from questionnaires 28 as well as from the wearable sensor data (smartwatch or ring) and trigger a PRO module/domain for reporting sleep quality measures.

More specifically, an Edge/Mobile Computing based Symptom Tracking Analytics services are preferably employed on a mobile phone thereby reducing the challenges of patient data privacy and security. Moreover, an interoperable PRO Bank, a centralized database running on a secure server, is preferably used. The digital platform 10 of the present invention preferably will query the PRO domain based on the context derived from the wearable data analytics. Billable, reimbursable digital health PRO services for doctors to make clinical decisions is also possible.

Because Parkinson's Disease affects uniquely to each patient, there currently exist many approved PROs including but not limited to NIH Toolbox, PDQ-39, PDQ-8, and PROMIS29. Since it may be challenging to settle on a single PRO module to have a comprehensive understanding of a patient's experiences, management of such a clinical decision is possible with the present invention. For example, it is possible to link PROs to electronic health records (EHRs) and generate specific end-points to achieve improved quality of care for improved patient satisfaction for increased revenue. The integration of PROs with an EHR system will enhance adherence and monitoring with prescribable digital health services.

Still further, the system and method 10 of the present invention may deploy disease-specific education from PRO and symptom tracking using trusted resources for disease and symptom-specific education. Since patients with Parkinson's Disease face a variety of symptoms and experiences for which they have limited understanding, the present invention 10 can develop a collective knowledge base from reliable resources and can offer PRO guided education via the present invention.

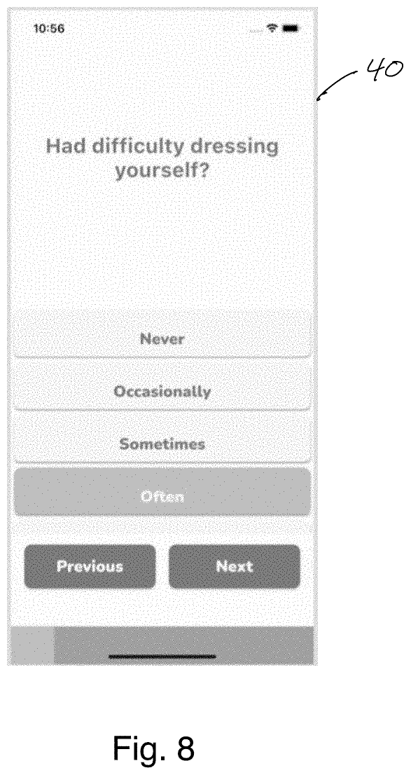

Schedulable micro-assessments 40, as seen in FIGS. 4 and 8, are also a component of the present invention 10. Thus, neurologists can gain an added understanding of how their patients are doing at home. The present invention can provide personalized PROs to the neurologists for enriching patient-specific treatments. The system and method 10 of the present invention can provide a machine learning-based scoring system to quantify health measures and PRO outcomes for the healthcare providers, as further outlined below.

More specifically, the present invention 10 further provides a new and novel system to monitor neurodegenerative disorders and diseases through assessments in daily life settings that uniquely combines both non-motor and motor factors in its determination of the disease state. Such a combination of non-motor factors 14 and motor factors 16 for the determination of the disease state is not found in the prior art.

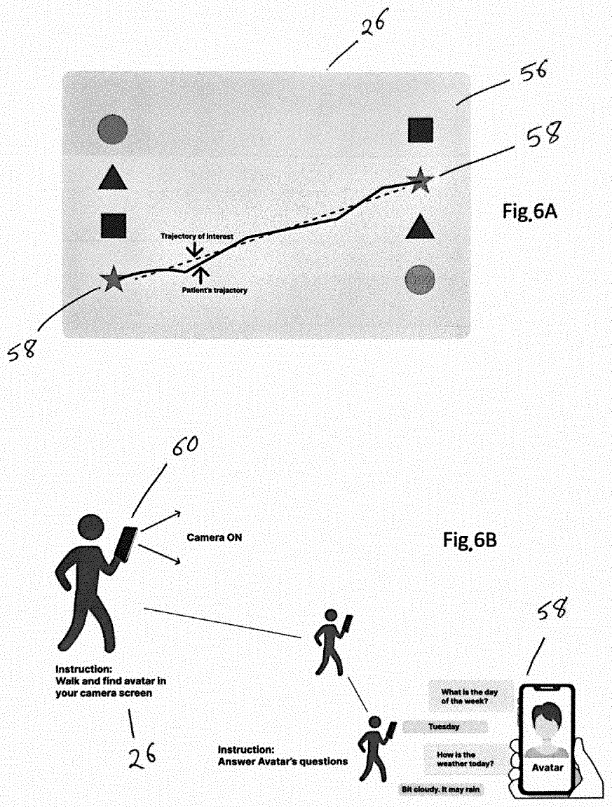

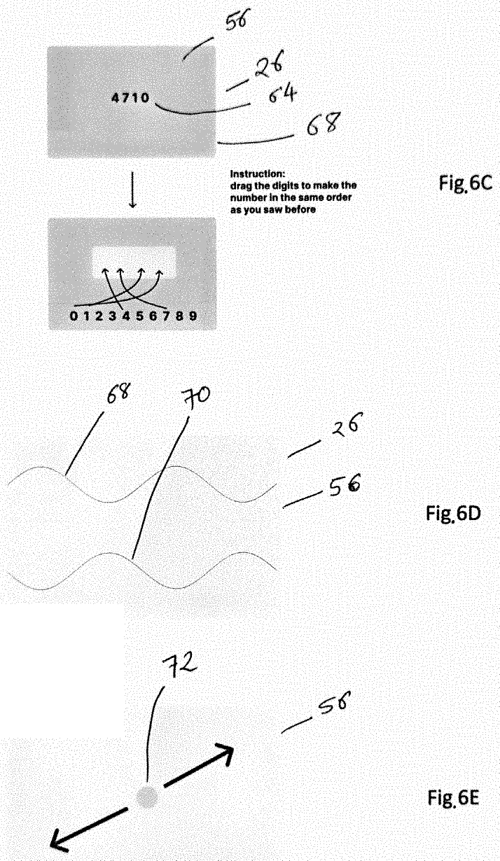

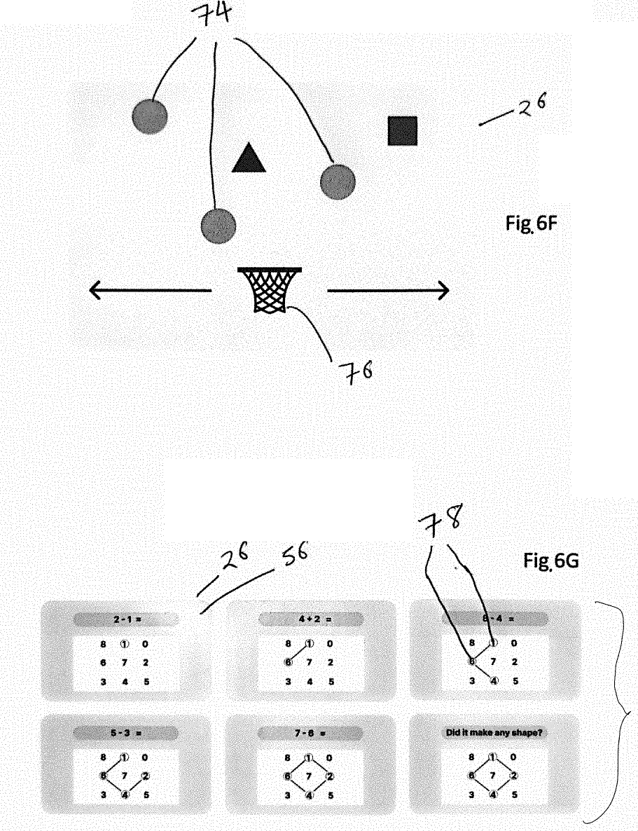

Referring now to FIGS. 6A-G, one or more tasks 26 are given to the patient to be completed to provide benchmark assessments. The form and factor of a given task and subtasks 26 may be altered to remain novel to the user overtime. The ensemble of the desired tasks or subtasks 26 may be adapted to minimize interference across components by means of statistical analysis. The tasks 26 to be completed by the patient may be directly solely to either a non-motor factor 14 or a motor factor 16. Or, it is possible that a single task 26 may be designed to assess both non-motor factors 14 and motor factors 14 simultaneously.

Also, the assigned and completed tasks 26 can be driven by prior knowledge of the user and or other task managers. The users of the system and method 10 of the present invention may include the patient with the disorder, the caregiver 44 or partner of the patient, and the overall health care team.

In accordance with the present invention, a given task 26 for benchmark assessment may include various individual components, such as 1) Items—which may include variation in shapes/contours/patterns/shading/hue/color/brightness of, for example, icons or other visuals presented; 2) Pathways—which may include variation such as in contour/degrees of freedom/visibility; 3) Instructions—which include variation in language/speed/tone/repetition/timing/brevity; and 4) Data Sources—which is collected from the patient 49 with the disorder or disease, the caregiver 44 or partner, the healthcare team 46, and/or ambient sensors, such as mobile, positioning or wearable devices.

Referring back to FIG. 1, it should be understood that the reported outcomes may be of any type and still be within the scope of the present invention. For example, in practice, there could be, for example, four types of reported outcomes. These include patient reported (patient only) 49, observer reported (non-clinical reporter such as a care-partner or other individuals without clinical experience) 44, clinical reported (same observer but requiring a qualified clinician to fill out the report, which are important as they help inform the capacity measure) 46, and device reported where sensors fill out the report.

The structure of the components involved in a given task 26 may also include data streams (such as from sensors, including ambient sensors and user engagement with a given interfaces, such as those of a digital device in the form of a tablet, or the like) of parameters such as active energy burned, heart rate, body temperature, resting heart rate, respiratory rate, steps, walking heart rate, distance walking/running, flights climbed, elevation gain, minutes moved, distance changes, sleep durations, exercise time, high/low heart rate events, heart rate variability, point/path variability, response times, completion rates, accuracies in task completion, and the like. The completion of a given task 26 by the patient 49 provides outcome measures that are used to determine the overall state of the given disorder or disease, as represented by a single score 42.

More specifically, in accordance with the system and method of the present invention, the knowledge for the non-motor 14 and motor 16 outcome measures are derived from informational components such as: 1) Cognitive 18—Attention, computation, executive functioning, learning/memory, orientation, language, processing speed and digit span; 2) Emotional 20—Anxiety, depression, apathy, fatigue, quality of life; 3) Behavioral 24—Activities of daily living, Sleep, Social, Intellectual; and 4) Physical 22—Gastrointestinal, urinary, mood, pain, fatigue, speech/swallowing and activities of daily living, tremor, kinesia, rigidity, hand-eye coordination, gait/stability, and freezing.

Tasks 26 are assessed through measures to score a unique daily function through the discovery and analysis through a combination of various elements.

Trusted third-party observations are employed to derive a measure of maximal effort or benchmarks, enabling prior observations may provide an initial baseline measure. These third-party observations may be made by clinical, digital, or by other fashion.

A measure of perceived ability (perception) 48, as seen in flowchart map of FIG. 4, is derived from patient/person-reported questionnaires 28, scales, ecological momentary assessments 40, or open-ended text or audio recordings. Perceived ability 48 can be measured with the use of tools to monitor user engagement during short assessments 40 in the form of tasks 26 that can measure non-motor 14 and/or motor 16 capability. For example, monitoring the patient's engagement with the interaction with presented items such as buttons, keyboard typing, tapping, microphone, haptic vibration, and other user interface (UI) components that can help inform both motor 16 and non-motor 14 informational components, which uniquely are combined together to arrive at a single score 42 representative of the state of the disease or disorder. Statistical/AI models are preferably integrated throughout the touch inclusive portions of the patient's engagement with a device to monitor movement disorders, such as tremors (shaking that occurs at rest), slow movements, stiffness of arms, legs, and trunk, problems with balance and tendency to fall. The statistical/AI models are integrated to monitor voice quality during the oral engagement portions of the users engagement to monitor voice changes, such as jitter/shimmer, loudness, fundamental frequency, and the like. The present invention 10 allows for variations in assessment or task types to enable estimations of maximal effort or capacity (gathered through occasional activity that requires maximal functioning to complete), performance abilities (derived from one or more mobile, wearable, ambient or environmental sensors), along with perceived abilities (based on presenting validated questionnaires for their known conditions, scales, ecological momentary assessments, or open-ended text or audio recordings via the interface). Thus, statistical or AI-models are used to process scores of capacity using maximal effort, performance using measures of active daily living and perception 48 using measures of perceived ability.

Referring again to FIGS. 6A-G, different tasks 26 with both non-motor factors 14 and motor factors 16 are carried out by the patient 49 so various assessments may be measured for the calculation of a daily function score 42. For example, in FIG. 6A, preferably on a digital device with a touch screen 56, the patient 49 drags a shape 58 from the left side of the screen to the right side of the screen to keep the shapes 58 in the same category. The dragging operation is preferably conducted multiple times, such as five, so multiple assessments can be measured. For example, for this task 26, tremors can be measured from the monitoring of fine motor dragging to detect jitter, for example. Processing speed is measured in this task through the time it takes to drag each shape 58. Planning is also measured by determining whether the shape 58 is moved across to the correct location. Attention can also be measured by conducting the task multiple times, such as five times. Thus, non-motor factors 14 and motor factors 16 are measured at the same time using a single task 26.

In another example, as shown in FIG. 6B, a task 26 may be directed to gait and speech. In this task, a patient 49 is asked to find an avatar 58 on a camera 60 in the room and then to walk to the avatar 58, which includes walking steps and turns. Sensors on the patient 49 are used, such as accelerometers, gyroscopes, camera, microphones, and the like are preferably employed. The patient 49 talks to the avatar 58 and answers questions. Thus, with this assessment task 26, gait parameters are measured to detect, for example, walking speed. The assessment measure of Planning identifies an object and reach. The assessment measure of orientation is present to measure time and space. Language assessment is also present in this task where the understanding of language, in the form of questions and instructions, is measured. The loudness, jitter, fundamental frequency of speech is also another assessment measure that is achieved with this task.

In another example, in FIG. 6C, a digital span task 26 may be used where the patient 49 is shown a number on a first screen 56 and then on a second screen 64, the patient is asked to drag the digits 66 in the box 68 in the same order as the indicated number. Thus, with this task 26, digital span/accuracy is measured to assess the remembering of numbers 66 in a given sequence. Also, processing speed is assessed for the time it takes for the patient to recall the numbers 66.

FIG. 6D shows a two finger motor task 26 where the patient 49 touches and moves a pointed finger on one line 68 and a thumb on another line 70 on the screen. In FIG. 6E, the patient also zooms at 72 in on the display 56 with two fingers on a map or picture, for example. This test addresses the assessment measure of tremors and kinesia (detecting fine motor jitter deviation from a line) and the assessment of rigidity as a large deviation from time.

Also, FIG. 6F shows another task 26 involving ball catching where the patient 49 is asked to catch only the ball-shaped falling objects 74 in/with the basket 76. The basket 76 is dragged left and right by the patient 49 using a finger on a screen 56 of a device, such as a tablet. Tremors/kinesia are detected using the finger movement left and right as shown by the arrows. Planning is measured to see how the patient 49 plans the move of the basket to catch the balls. Attention is measured by determining if the patient 49 is catching ball-shaped objects 74 only. Processing speed is determined for the time it takes to drag the basket back and forth.

Still further, FIG. 6G shows yet another task 26 in the form of a computation. In this task, the patient 49 is presented with a simple math computation. The patient 49 is asked to connect the dots 78 on a grid as they solve the computation equation. At the end, the task 26 asks if it made any shapes. The assessment measure of computation is present to determine if the patient can compute math. Shape recognition determines whether the patient can recognize shapes from the formed dots. As with the other tasks 26, processing speed is measured to understand how long it took for the patient 49 to make the math computation.

A measure of active daily living is derived from wearable/environmental/social datasets about mobility, health and wellness of the patient. For example, any device 34 may be used for this purpose, such as a smart watch, smart phone, activity tracker, and the like, as shown in FIG. 7. Moreover, these wearable devices 34 may be adapted to include measures specific to patients 49 who exhibit symptoms of neurodegenerative diseases including, but not limited to, Parkinson's disease, Alzheimer's disease, Huntington's disease, amyotrophic lateral sclerosis (ALS), motor neuron disease, ataxia, multiple system atrophy (MSA), and the like. The monitoring of these measures through distributed datastores may utilize blockchain technology for added security, privacy, tracking and reliability.

In view of the foregoing, the method of the present invention quantifies the severity of a subject's neurodegenerative disorder. Questionnaires, benchmark tests are carried out and sensors are used for continuous monitoring. Short assessment tests of the subject are conducted. The data collected, which includes non-motor outcome measures and motor outcome measures from the foregoing are aggregated to arrive at a single score that quantifies the severity of a neurodegenerative disorder of the subject based on the output dataset.

For example, the qualification of scoring is a process of information mining via the statistical inference and the intercorrelation of the data acquired from the various sources including task performance on the digital interface, questionnaires, and sensor data. The scoring algorithm consists of a set of mathematical functions and models for signal processing, machine learning and AI, allowing to measure the disease state of patients with neurodegenerative disorder in single and multiple domains including motor and non-motor. The machine learning model is trained over time using the received data from different sources and used for the longitudinal monitoring of the disease state in daily life settings. The score is a measure of performance, perception, and capacity from the acquired data that are not only limited to the patients themselves but also caregivers/carepartners and clinicians. The correlation of the data from patients, caregivers, and clinicians is a part of the scoring process as it gives a deeper insight on the disease state of patients. For example, the mathematical functions that can be used for the scoring model of the present invention include normalization; detrending; principal component analysis; maximum likelihood estimators for the domains; match filtering is used based off of the domain variance, means, frequencies and powers are characteristics used in the match filtering; inter correlation or covariance used in ensuring the signals are properly decoupled; and the like.

It would be appreciated by those skilled in the art that various changes and modifications can be made to the illustrated embodiments without departing from the spirit of the present invention. All such modifications and changes are intended to be covered by the appended claims.

What is claimed is:

1. A method for quantifying the severity of a subject's neurodegenerative disorder, comprising the steps of:
providing a subjective questionnaire to a subject, the answers to which resulting in a patient-reported outcome dataset;
performing a benchmark test of the subject by the subject performing a task resulting in a task result dataset;
providing a device to a subject, the device including at least one sensor configured and arranged for continuous sensing at least one parameter of the subject and/or an environmental condition proximal to the subject; collecting data sensed by the at least one sensor resulting in a sensor dataset;
conducting a short assessment test of the subject resulting in a short assessment dataset;
aggregating the patient-reported outcome dataset, task result dataset, sensor dataset, and short assessment dataset into an output dataset including non-motor outcome measures and motor outcome measures; and
generating a single score quantifying the severity of a neurodegenerative disorder of the subject based on the output dataset.

2. The method of claim 1, wherein the at least one parameter is active energy burned, heart rate, body temperature, resting heart rate, respiratory rate, steps, walking heart rate, distance walking/running, flights climbed, elevation gain, minutes moved, distance changes, sleep durations, exercise time, high/low heart rate events, heart rate variability, point/path variability, response times, completion rates and accuracies in task completion.

3. The method of claim 1, wherein the environmental condition is ambient temperature, weather, imagery, and social.

4. The method of claim 1, wherein the sensor is a wearable sensor.

5. The method of claim 1, wherein the sensor is an ambient sensor.

6. The method of claim 1, wherein the task is directed to shapes, contours, patterns, shading, hue, color, brightness, degrees of freedom, visibility, language, speed, tone, voice, repetition, timing, brevity.

7. The method of claim 1, wherein the non-motor outcome measures and motor outcome measures are derived from informational components selected from the group consisting of: cognitive, emotional, behavioral, and physical.

8. The method of claim 1, wherein the non-motor outcome measures are derived from at least one of the domains of attention, computation, executive function, learning/memory, orientation, language, processing speed, and non-motor aspects of experiences of daily living.

9. The method of claim 1, wherein the motor outcome measures are derived from at least one of the domains of motor aspects of experiences of daily living, motor examination, and motor complications.
